(19) United States
(12) Patent Application Publication
(10) Pub. No.: US 2018/0021592 A1
(43) Pub. Date: Jan. 25, 2018

(54) HAIR GROWTH STIMULATING BAND

(71) Applicant: Kim Segal, Plano, TX (US)

(72) Inventor: Kim Segal, Plano, TX (US)

(21) Appl. No.: 15/617,153

(22) Filed: Jun. 8, 2017

Related U.S. Application Data
(63) Continuation-in-part of application No. 15/598,747, filed on May 18, 2017.
(60) Provisional application No. 62/338,073, filed on May 18, 2016.

(51) Int. Cl. A61N 5/06

(52) U.S. Cl. CPC A61N 5/0617; A61N 2005/0647; A61N 2005/0659; A61N 2005/0651; A61N 2005/067

(57) ABSTRACT

The current invention includes a device and method for the promotion and stimulation of hair growth using one or more light sources such as a diode laser, each light source operating at a low wattage, collectively less than about 1000 mw, with the power level typically being in the region of 500 mw, in the infrared range at wavelengths in a range from about 2500 nm to about 10,000 nm. A diode laser operating in this range will have a greater dispersion rate than heretofore, thus requiring fewer diodes to cover the same area of scalp with less power required per diode laser.

### BACKGROUND OF THE INVENTION

This invention generally relates to human hair growth and, more particularly, to methods and devices for stimulating hair growth through stimulation of the hair follicles by means of a laser.

Alopecia (hair loss) is a major concern for the adult population. Expenditures for hair restoration products and treatments for hair loss represent a major component of the multibillion-dollar cosmetic industry in the United States. Examples of techniques for hair retention and regeneration include the use of hair weaving, the use of hairpieces, the application of hair thickening sprays and shampoos, hair transplantation, and the fashioning of coiffures which distribute hair to cover balding regions of the scalp. In addition, topical drug therapies, such as Minoxidil (Rogaine®) or oral drug therapies such as Finasteride (Propecia®), are in current use to stimulate hair growth in men suffering from male pattern baldness, i.e. baldness occurring at the crown and temples. However, this chemical cannot be used by women, can cause a negative skin reaction on the scalp, and is, therefore, not suitable for everyone, and efficacy is limited and not universal.

Diode laser systems have been developed for various medical treatments of the human body. See for example, Applicant's prior U.S. Pat. Nos. 5,755,752 and 6,033,431, which are both incorporated herein by reference in their entirety. Depending on the type of treatment desired, lasers of various wave lengths, periods of exposure and other such influencing factors have been developed.

Lasers are the newest surgical tool for the medical profession because laser light, as a result of its monochromatic and coherent nature, can be selectively absorbed by living tissue. The absorption of the optical energy from laser light depends upon certain characteristics of the wavelength of the light and properties of the irradiated tissue, including reflectivity, absorption coefficient, scattering coefficient, thermal conductivity, and thermal diffusion constant. The reflectivity, absorption coefficient, and scattering coefficient are dependent upon the wavelength of the optical radiation. The absorption coefficient is known to depend upon such factors as interband transition, free electron absorption, grid absorption (photon absorption), and impurity absorption, which are also dependent upon the wavelength of the optical radiation.

In living tissue, the predominant water component an absorption band determined by the vibration of water molecules. In the visible portion of the spectrum, there exists absorption due to the presence of hemoglobin. Further, the scattering coefficient in living tissue is a dominant factor.

Thus, for a given tissue type, the laser light may propagate substantially unattenuated through the tissue, or may be almost entirely absorbed. The extent to which the tissue is heated and ultimately destroyed depends on the extent to which it absorbs the optical energy and the power associated with the energy. It is generally preferred that the laser light be essentially transmissive through tissues which are not to be affected, and absorbed by tissues which are to be affected. For example, when applying laser radiation to a region of tissue permeated with water or blood, it is desired that the optical energy not be absorbed by the water or blood, thereby permitting the laser energy to be directed specifically to the tissue to be treated. Another advantage of laser treatment is that the optical energy can be delivered to the treatment tissues in a precise, well defined location and at predetermined, limited energy levels.

Ruby and argon lasers are known to emit optical energy in the visible portion of the electromagnetic spectrum, and have been used successfully in the field of ophthalmology to reattach retinas to the underlying choroidea and to treat glaucoma by perforating anterior portions of the eye to relieve interoccular pressure. The ruby laser energy has a wavelength of 694 nanometers (nm) and is in the red portion of the visible spectrum. The argon laser emits energy at 488 nm and 515 nm and thus appears in the blue-green portion of the visible spectrum. The ruby and argon laser beams are minimally absorbed by water, but are intensely absorbed by blood chromogen hemoglobin. Thus, the ruby and argon laser energy is poorly absorbed by non-pigmented tissue such as the cornea, lens and vitreous humor of the eye, but is absorbed very well by the pigmented retina where it can then exert a thermal effect.

Another type of laser which has been adapted for surgical use is the carbon dioxide (CO₂) gas laser which emits an optical beam that is well absorbed by water. The wavelength of the CO₂ laser is 10,600 nm and therefore lies in the invisible, far infrared region of the electromagnetic spectrum. It is absorbed independently of tissue color by all soft tissues having a high water content. Since it is completely absorbed, the CO₂ laser makes an excellent surgical scalpel and vaporizer since its depth of penetration is shallow and can be precisely controlled with respect to the surface of the tissue being treated.

Another laser in widespread use is the neodymium doped yttrium-aluminum-garnet (Nd:YAG) laser. The Nd:YAG laser has a predominant mode of operation at a wavelength of 1064 nm in the near infrared region of the electromagnetic spectrum. The Nd:YAG optical emission is absorbed to a greater extent by blood than by water making it useful for coagulating large, bleeding vessels. The Nd:YAG laser has been transmitted through endoscopes for treatment of a variety of gastrointestinal bleeding lesions, such as esophageal varices, peptic ulcers, and arteriovenous anomalies.

The foregoing applications of laser energy are thus well-suited for use as a surgical scalpel and in situations where high energy thermal effects are desired, such as tissue vaporization, tissue cauterization, and coagulation.

Although the foregoing laser systems perform well, they commonly generate large quantities of heat and require a number of lenses and mirrors to properly direct the laser light and, accordingly, are relatively large, unwieldy, and expensive. As such, they are unsuitable for use in stimulating hair growth.

Lasers are in increasing use to effect hair removal. This is done by overheating the hair follicles to destroy them. Recently, laser treatment has now been developed specifically for use as a positive stimulating agent for hair growth. The alleged key is to use low power lasers, so as not to destroy, but stimulate the follicles. Several patents have addressed this solution in different way. For example, see U.S. Pat. Nos. 6,497,719, 6,666,878, and 6,802,853. A commercial system similar to that disclosed in the '878 patent uses an array of circumferentially-spaced parallel rows of laser diodes in a hair-dryer type apparatus which rotates. These diodes are carefully arrayed in adjacent rows of staggered diodes to assure overlapping of the light fields of adjacent diodes. The prescribed diodes have a wave length of 400 to 1300 nm (670 nm preferred) and a power sufficient to generate a power density of 7-8 joules/cm². The various embodiments require dozens or even hundreds of diodes. These commercial units are quite expensive and retail for $25,000-$30,000, which severely limits its market and consequent availability to the public for hair growth treatment. Furthermore, these units are cumbersome and take up a significant amount of space within which to operate.

As can be seen, there is a need for a simpler, lower cost, and more space efficient system and method for stimulating hair growth with laser energy without damaging the tissue from the thermal effects of the laser energy.

### BRIEF DESCRIPTION OF THE DRAWINGS

The invention will be better understood and objects other than those set forth above will become apparent when consideration is given to the following detailed description thereof. Such description makes reference to the annexed pictorial illustrations, graphs, drawings and appendices.

FIG. 1 shows a device for treating patients for hair growth stimulation, according to an embodiment of the invention.

FIG. 1B shows a block diagram of a hair growth stimulating device according to one embodiment of the present disclosure;

FIG. 1C shows a block diagram of a hair growth stimulating device according to one embodiment of the present disclosure;

FIG. 2A shows an end view of a hair growth stimulating device according to one embodiment of the present disclosure;

FIG. 2B shows an end view of a hair growth stimulating device according to one embodiment of the present disclosure;

FIG. 2C shows a cross-sectional view of a hair growth stimulating device according to one embodiment of the present disclosure;

FIG. 3A shows a side view of a hair growth stimulating device according to one embodiment of the present disclosure; and

FIG. 3B shows an end view of a hair growth stimulating device according to one embodiment of the present disclosure.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

It is known in a commercially-available hair growth stimulation device to provide laser diodes having a wavelength of about 670 nm, activated at an undisclosed wattage. Applicant's prior patents disclose the use of a laser having wavelengths of from about 1,064 nm to about 2,500 nm for medical treatments that do not involve hair growth stimulation. It has been subsequently discovered that laser diodes having a wavelength within the region from about 2500 nm to about 10,000 nm can also be used for the stimulation of hair growth and tissue regeneration, and more specifically wavelengths in the region from about 2500 nm to about 5000 nm, and even more specifically wavelengths of about 3150 nm.

Broadly, the current invention includes systems, devices, and methods for a light source, typically a diode laser, operating in the infrared range at wavelengths of greater than about 2,500 nm and at a low total wattage, preferably less than about 1,000 mw for the total output of the device, and more preferably less than about 500 mw. A laser operating in this range will have a greater dispersion rate than heretofore, thus requiring fewer diodes to cover the same area of scalp stimulation for promoting hair growth. A number of factors govern effective scalp stimulation: laser diode wavelength and power (diode wattage); light beam divergence and dispersion; duration period of laser light application/stimulation; rate of application, i.e. the number of periods per unit of time; and the distance between the diodes and the scalp. While prior art devices provide a substantial space between the diodes and the scalp, the Applicant has found that a minimal spacing may be more effective when using diodes in this infrared range and at low wattage.

Unlike prior patents where the diodes are arranged in a pattern on a cap placed over the patient's head, the inventive device may feature a circumferential band that extends over the surface of the patient's head from ear to ear, where a distance between the band and the scalp is maintained at all points within a known tolerance range. Along an inner surface of the band, one or more diode lasers may be positioned to shine in a direction towards the scalp. This band may be pivotally moved over the surface of the scalp within a range from about the nape of the neck to about the forehead of the patient. By controlling the extent of travel of the band over the scalp surface, the power intensity of the diode lasers, and the on/off status of the diode lasers, different areas of the scalp may be targeted for radiation while leaving other areas of the scalp alone. Additionally, multiple bands may be configured at different angular displacements from each other and rotationally moved as a unit over the scalp. If the distance between the diode lasers on a particular band is made to vary from band to band, a more complete coverage of the scalp may be obtained.

This device may find application in the medical fields for encouraging hair growth. It may also find application for appearance enhancement in the cosmetic industry.

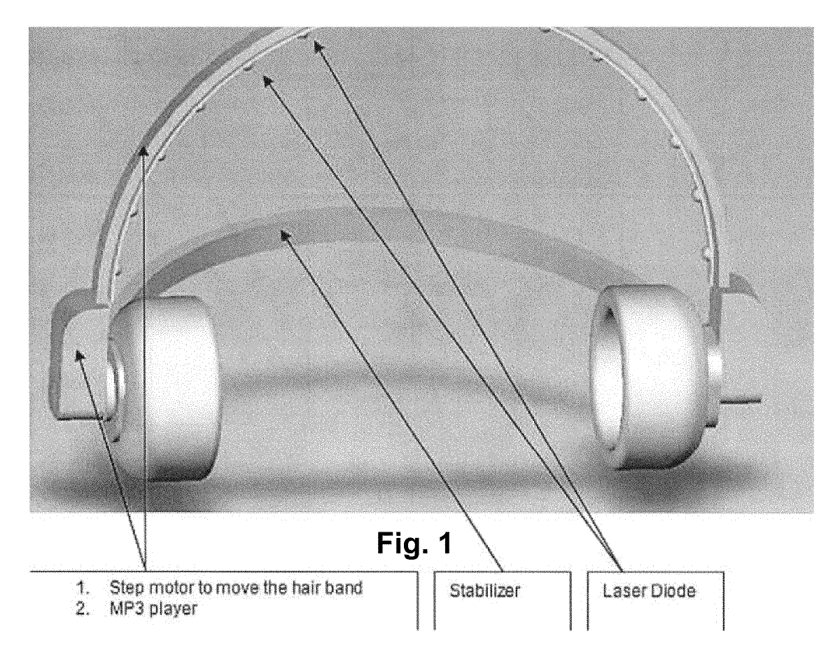

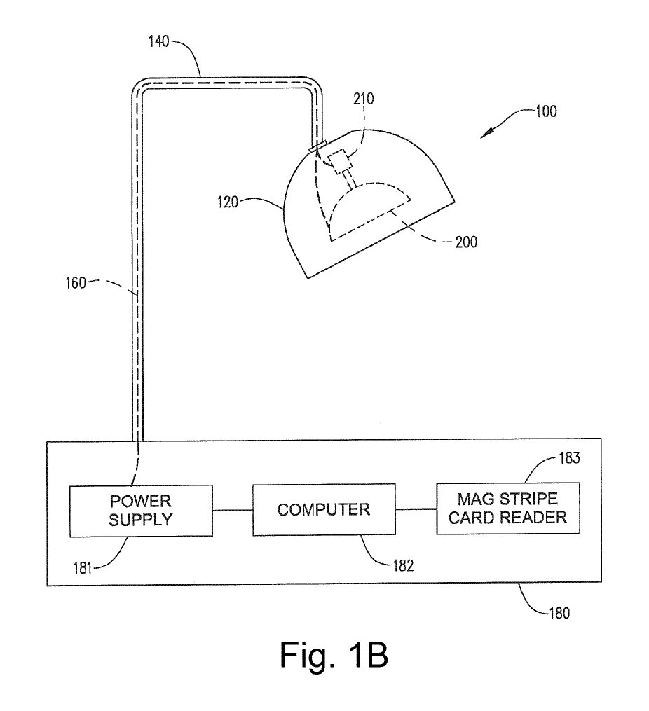

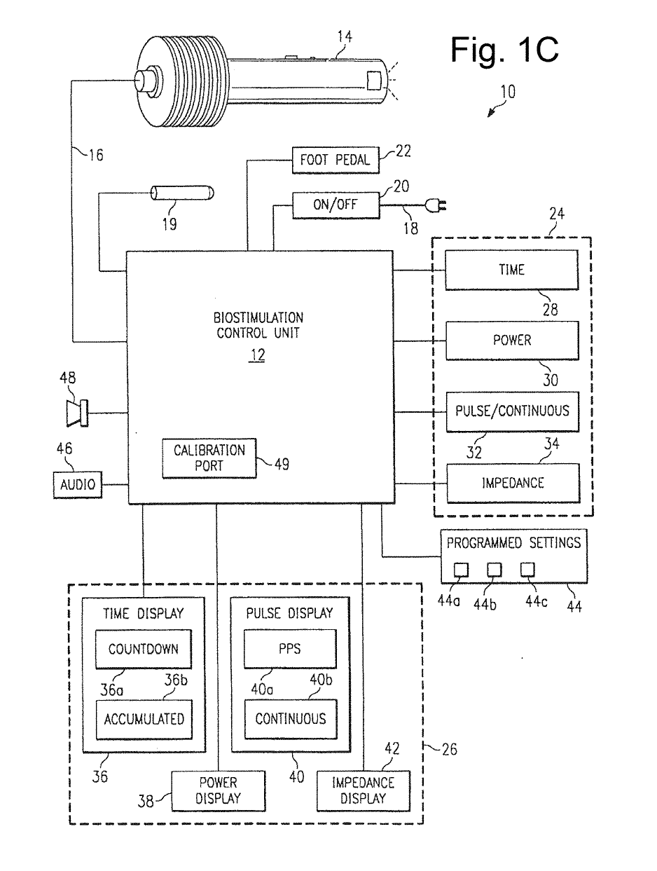

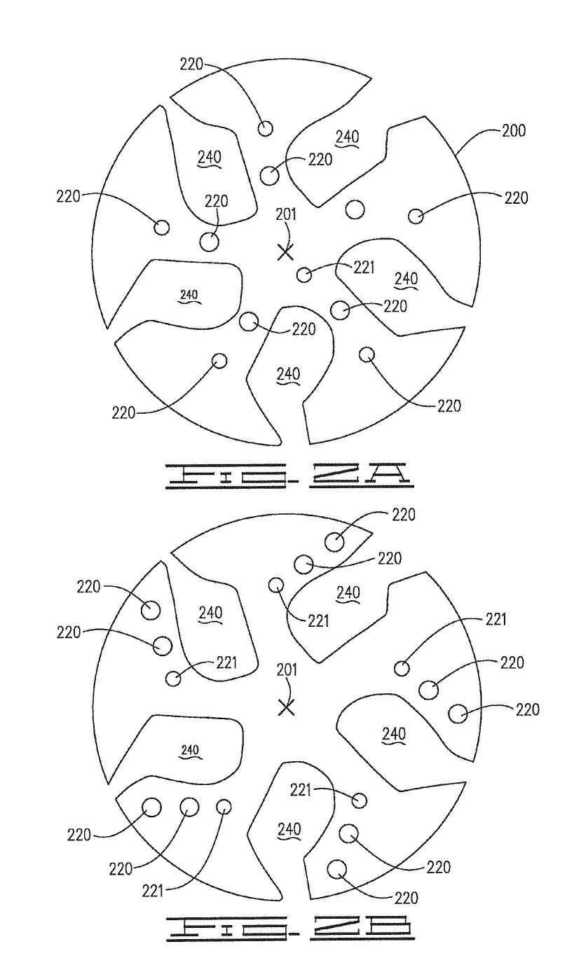

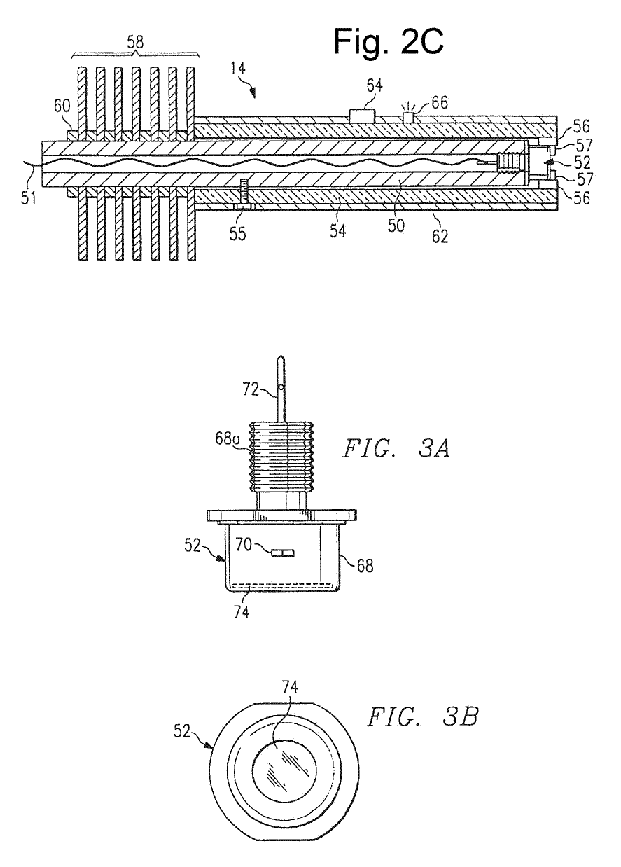

Referring now to FIG. 1, a hair band stimulation device 100 is shown, according to an embodiment of the invention. A pair of ear cups may be fixedly positioned over the ears and maintained at a constant angular displacement about the head by a stabilizing means. The ear cups may comprise a stepping motor that moves a band over the scalp in a controlled manner. The moveable band may have diodes along its inner surface for providing radiation against selected portions of the scalp.

It is also contemplated that the current invention and ranges may be utilized with other existing devices such as but not limited to that disclosed in U.S. Pat. No. 7,722,656 and U.S. Pat. No. 6,033,431.

As shown in FIG. 1, the stabilizing means may be a solid band about the back of the head that compressively maintains the ear cups over the ears without rotating the ear cups. The stabilizing means may additionally comprise supports (not shown) and other devices that will position the ear cups against the shoulder and other body parts. The stabilizing means may thus provide a fixed frame of reference within which an angular rotation of the band may take place. The band shown in FIG. 1 may be exemplary of other stabilizing means and should not be taken as limiting the invention to the embodiment shown in the figure.

Each ear cup may contain a stepping motor for moving the moveable band over the scalp; however, a single stepping motor may be used on a single ear cup with the other ear cup providing a rotational bearing facilitating angular movement of the moveable band, without departing from the inventive concept. The ear cup may also contain electronic means for providing music, radio, instructions to the patient, and other audio sources to the patient's ears in order to entertain the patient during the radiation process. The ear cup may also have a soft cushion to prevent discomfort during the radiation process.

The moveable band may contain one or more diodes along its inner surface, each diode being positioned to shine in a direction that is more or less perpendicular to the scalp surface. If two or more diodes are configured, then the distances between adjacent diodes may be equal to each other or the distances between any pair of adjacent diodes may be different from the distance between any other pair of adjacent diodes, without departing from the scope of the invention. The diodes configured within the moveable band may provide near infrared radiation having a wavelength that is with a region from about 2500 nm to about 10,000 nm, and more preferably within a region from about 2500 nm to about 3500 nm, and even more preferable about 3150 nm. It is also contemplated to utilize 1350+/−20 nm and up to 2500 nm. Thus, the wavelength can be anywhere from 1350+/−20 nm up to 10,000 nm. It is still further understood that greater and less is contemplated.

Those of skill in the art will understand and appreciate that the above specific frequency ranges are provided only by way of example, and that light sources able to emit light anywhere within the range between approximately 1,330 nm and approximately 10,000 nm may be employed in certain embodiments of the present disclosure. It is possible that frequencies below 1,330 nm may be employed in certain embodiments. It is also possible that frequencies above 10,000 nm may be employed in certain embodiments. Certain embodiments may employ two or more light frequencies, which may be within or outside of the above-referenced frequency ranges.

Each diode may be operated at a power level of from about 0 mw to about 100 mw, with the total power level applied to all diodes on the band being no more than 1000 mw. The power level applied to each diode may be independently controlled without affecting the power level applied to other diodes, without departing from the scope of the invention.

Although FIG. 1 shows a single moveable band, multiple bands may be configured for angular movement over the scalp by the ear cups. Each band may preferably have a spacing between diodes that differs from the spacing for other bands, in order to provide more complete coverage of the scalp. The moveable bands may be configured with a constant angular displacement from an adjacent moveable band, with all bands moving as a unit.

Although the principal embodiment described herein employs laser diodes as an example light source, there is nothing within the spirit and scope of the present disclosure limiting the light sources to laser diodes, specifically. Depending on the specific application, light may be generated via a variety of laser types, including but not limited to gas lasers, chemical lasers, dye lasers, metal-vapor lasers, solid-state lasers or semiconductor lasers. It is not necessary that the light used in the present disclosure be generated by a laser. A variety of suitable light sources may be employed in the present disclosure, as will be known to, and appreciated by, those of skill in the art. Further, any suitable devices capable of generating, shifting, refracting, reflecting, polarizing, diverting, focusing or filtering light in such a manner as to provide light at the correct location within the proper frequencies and at the proper level of intensity may be used to generate and direct light in connection with the embodiments disclosed herein. These devices may include, but are not limited to, fiber optics, conduits, mirrors, lenses, prisms and filters.

A wiring harness 160 may be provided to connect the hair band stimulation device 100 and a controller 180. The controller 180 may provide both control and power to components contained within the hair band stimulation device 100. In this respect, the controller 180 be identical to other such controllers shown in the prior art, e.g. U.S. Pat. No. 7,722,656, issued May 25, 2010, to Segal, and incorporated herein in its entirety.

The controller 180 may be adapted to accepted parameters selected by the operator, such as speed of movement of the band, angle of rotation, direction (forward or back), actuation of the diodes (i.e. points of time at which a particular diode may be turned on or off) and power level of each diode on each band. This set of parameters may be termed a cyclical sequence. The cyclical sequence may be stored in the controller 180 for convenience. A cyclical sequence may be developed for different patterns of hair loss, stored within the controller 180, and retrieved as needed, depending upon the patient.

One example of a diode used according to the invention may be a Boston Electronics Model LED34-05, having a window cap that is 3.5 mm in diameter (approx. 0.15 in.). This diode 220 has a peak emission wavelength of 3400 nm (3.4 microns) and a maximum emissive power of 20 μw at 2.5% duty cycle in pulsed mode. However, such diodes of this type may also be operated in continuous mode without departing from the scope of the invention. Diodes of this type may operate at a power level of up to about 100 mw individually, but nominally it is expected that 20 to 30 diodes, each operating at a power level of between about 0 mw to about 15 mw, would be a typical configuration for the invention, with the total wattage expended for all diodes collectively being less than or equal to about 500 mw. The beam divergence/dispersion of this diode may be controlled by means of a lens in the top of the cap surrounding the diode. A lens will exhibit the narrowest dispersion, while a diode cap having no lens will exhibit intermediate dispersion and a capless diode will exhibit the widest dispersion. The divergence/dispersion pattern chosen may be dependent upon the distance between the surface of the scalp and the diode, so that sufficient coverage of the scalp area may be achieved.

The light sources of the inventive device described herein for stimulating hair growth may typically be operated at a collective power level of about 500 mw or less. However, there may be certain circumstances where a higher power level is warranted. For example, in the case of cancer patients, the chemotherapy used to treat the cancer will frequently result in hair loss. Such patients have been found to require higher levels of hair follicle stimulation than the normal patient population. These higher levels of stimulation may be provided by power levels that exceed 500 mw for the collective laser light sources but generally not exceeding 1000 mw collectively.

The apparatus thus described may be used to promote hair growth from the scalp of a patient according to a method of the invention. According to the method, one or more of the diodes may be arranged along the inner surface of the band according to a fixed pattern. A periodic cycle may be programmed into the controller 180 that actuates the band and diodes, which will cause the band to move in a repeated periodic movement over the scalp. The power supplied to each diode may be from about 0 mw to about 15 mw, so that the total power supplied to all diodes does not exceed 500 mw. The band may then be allowed to periodically cycle through its programmed course for a fixed length of time. Multiple treatments of this type may be necessary to complete the hair stimulation process.

Thus as can be seen, the invention provides a device and method for the stimulation of hair growth using a multiplicity of diodes arranged on a band over the scalp, the diodes operating at longer wavelengths and at lower power than heretofore. It should be understood, of course, that the foregoing relates to exemplary embodiments of the invention and that modifications may be made without departing from the spirit and scope of the invention as set forth in the following claims.

I claim:

1. A method for promoting the hair growth on the head of a patient, the method comprising the following steps:
arranging one or more light sources along one or more circumferential bands about the scalp of the patient and at a distance between the band and the scalp, each light source emitting a wavelength of coherent light at a specified power level, the wavelength in a range from about 2500 nm to about 10,000 nm;
moving the one or more bands over the scalp according to a periodic cycle, the movement accomplished within a length of time; and
wherein a selected portion of the scalp is traversed by the dispersion pattern of the light sources at least once during the periodic cycle.

2. The method according to claim 1, wherein the specified power level of each light source is between 0 mw and 100 mw.

3. The method according to claim 1, wherein the total collective power level of the device is less than about 1000 mw.

4. The method according to claim 1, wherein the specified wavelength of each light source is about 3400 nm.

5. The method according to claim 1, wherein the light source is a diode laser.

6. A device for stimulating hair follicles of a scalp of person through exposure to coherent light, the device comprising:
a pair of earpieces, each earpiece positioned over the ear, the pair supported by a support means extending between the earpieces in a stationary position with relationship to the shoulders and neck of the person;
a band extending between the earpieces circumferentially around the head of the person for reciprocal motion between the nape of the neck and the forehead, the band positioned a distance away from and over the scalp, the band having one or more light sources adapted to emit a beam of coherent light in a direction of the scalp, each light source emitting coherent light having a wavelength in a range of from about 2500 nm to about 10,000 nm;
a means for stabilizing the band for angular movement over the scalp; and
a means for controlling the angular movement of the band and the actuation of the light sources.

7. The device according to claim 6, wherein the light source is a diode.

8. The device according to claim 6, wherein the specified power level of each light source is between 0 mw and 100 mw.

9. The device according to claim 6, wherein the total collective power level of the device is less than about 1000 mw.

10. The device according to claim 6, wherein the specified wavelength of each light source is about 3510 nm.

11. The device according to claim 6, wherein the rotational movement is accomplished according to a cyclical sequence.
